(19) United States
(12) Patent Application Publication
(10) Pub. No.: US 2024/0277968 A1
(43) Pub. Date: Aug. 22, 2024

(54) MEDICAL DEVICE FOR STIMULATING NEURONS OF A PATIENT WHICH PREVENTS BLANK WINDOW STIMULATION

(71) Applicant: Gretap AG, Zug (CH)

(72) Inventor: Peter Alexander TASS, Tegernsee (DE)

(21) Appl. No.: 18/571,309

(22) PCT Filed: Jun. 21, 2022
(86) PCT No.: PCT/EP2022/066924

§ 371 Date: Dec. 18, 2023

Related U.S. Application Data
(60) Provisional application No. 63/212,738, filed on Jun. 21, 2021.

(51) Int. Cl. A61M 21/00

(52) U.S. Cl. CPC A61M 21/00; A61M 2205/05

(57) ABSTRACT

A medical device is provided for stimulating neurons of a patient to suppress a pathologically synchronous activity of the neurons. The device includes a non-invasive stimulation unit for administering different stimuli (s) to the patient's body via at least one stimulation channel and a control unit for selectively actuating the stimulation unit. The control unit actuates the stimulation unit to successively generate a first stimulus and a subsequent second stimulus in a sequence such that a minimum non-stimulation period (P_{cc}, P_{ic}) is provided between the first and the second stimulus. The control unit also sets the duration of the minimum non-stimulation period (P_{cc}, P_{ic}) in dependence on at least one stimulus characteristic of the first stimulus.

### TECHNICAL FIELD

The invention relates to a medical device for stimulating neurons of a patient to suppress a pathologically synchronous activity thereof.

### TECHNOLOGICAL BACKGROUND

Several brain disorders, such as Parkinson's disease, are characterized by an abnormally strong synchronous activity of a neuronal population, i.e. strongly synchronized neuronal firing or bursting. Besides Parkinson's disease, this may also apply, for example, to essential tremor, dystonia, dysfunction after stroke, epilepsy, depression, migraine, tension headache, obsessive-compulsive disorder, irritable bowel syndrome, chronic pain syndromes, pelvic pain, tinnitus, dissociation in borderline personality disorder and post-traumatic stress disorder. As such, abnormally synchronized neuronal activity is of great relevance for several neurological and psychiatric disorders.

For treating such brain disorders, non-invasive treatment approaches are known which apply, e.g., acoustic or vibrotactile stimulations to suppress abnormally synchronized neuronal activity. Specifically, according to one approach, periodic stimulations are administered to a patient which are intended to selectively activate at least a part of the patient's neurons affected by the abnormally synchronized activity. This stimulation may be performed according to a relatively simple and repeating stimulation pattern which specifies what kind of stimuli are administered to the patient and at which time. According to a further known approach, the non-invasive stimulation may be carried out according to a more complex actuation pattern which may continuingly vary during and among treatments as described, for example, in WO 2016/207247 A1 and WO 2019/243634 A1. Further, a stimulation technique referred to as “Coordinated Reset” (CR) is known which applies characteristic sequences of brief stimuli administered to different subpopulations within an abnormally synchronized neural network. By applying these stimulation techniques, a desynchronization of the targeted neural network may be restored and maintained.

The therapeutic success of such non-invasive treatment approaches may substantially depend on the stimulation pattern, i.e. characteristics and timing of the different stimuli to be administered to the patient's body. In other words, in order to ensure that the patient's neurons affected by the abnormally synchronized activity are effectively stimulated during treatment, the characteristics and timing of the different stimuli need to be set properly.

### SUMMARY OF THE INVENTION

It is thus an object of the present invention to provide an improved medical device for stimulating neurons of a patient to suppress a pathologically synchronous neuronal activity, which in particular enables to provide an effective treatment of neurological and psychiatric disorders caused by an abnormally synchronized neuronal activity.

This objective is solved by the subject matter of the independent claim.

Accordingly, a medical device is proposed for stimulating neurons of a patient to suppress a pathologically synchronous activity of the neurons.

The proposed medical device may be used for the treatment of neurological or psychiatric disorders or diseases which may be caused by a pathologically synchronous neuronal activity. In other words, the proposed medical device may be used to treat a pathologically synchronous activity of neurons of a patient. For example, the medical device may be used for the treatment of Parkinson's disease or tinnitus. Alternatively or additionally, the medical device may be used for the treatment of other neurological or psychiatric disorders or diseases, in particular for the treatment of at least one of essential tremors, dystonia, epilepsy, tremors as a result of Multiple Sclerosis as well as other pathological tremors, depression, movement disorders, diseases of the cerebellum, obsessive compulsive disorders, Tourette syndrome, functional disorders following a stroke, spastics, sleep disorders, schizophrenia, irritable bowel syndrome, addictive disorders, personality disorders, attention deficit disorder, attention deficit hyperactivity syndrome, gaming addiction, neuroses, eating disorders, burnout syndrome, fibromyalgea, migraine, cluster headache, general headaches, neuralgia, ataxy, tic disorder or hypertension, and also for the treatment of other diseases.

The aforementioned diseases can be caused by an impairment of the bioelectric communication of groups of neurons which are connected to one another in specific circuits. Hereby, a neuron population generates a continuous pathological neuronal activity and a pathological connectivity (network structure) possibly associated therewith. In this respect, a large number of neurons form synchronous action potentials, this means that the concerned neurons fire or burst excessively synchronously. In addition, the pathological neuron population may have an oscillating or intermittent neuronal activity, this means that the neurons fire or burst rhythmically or intermittently. In the case of neurological or psychiatric diseases, the mean frequency of the pathological rhythmic activity of the concerned groups of neurons approximately may be in the range of 1 Hz to 60 Hz, particularly in the range of 1 Hz to 30 Hz, but may also be outside of this range. By contrast, the neurons of healthy people fire or burst qualitatively differently, for example, in an uncorrelated manner.

In other words, each of the aforementioned diseases may be characterized by at least one neuronal population in the brain or spinal cord of the patient which has a pathological synchronous neuronal activity. For suppressing such a pathologically synchronous activity, the proposed medical device may be configured to stimulate the affected neuronal population so as to cause the affected neuronal population to fire or burst in an uncorrelated manner, i.e. non-synchronously.

Specifically, the medical device may be a non-invasive treatment device. This means that the medical device deploys a non-invasive procedure to achieve the intended therapeutic effect. In other words, in an operational state, the medical device may not be implanted into the patient's body and, hence, does not require skin incisions.

For acting upon the patient and thus for achieving the intended therapeutic effect, the medical device is equipped with a non-invasive stimulation unit, also referred to as ‘stimulation unit’ in the following, for administering different stimuli to the patient's body. In other words, the stimulation unit may be configured to generate the different stimuli selectively and intermittingly.

In the context of the present disclosure, the terms ‘stimulus’ and ‘stimuli’ refer to any object or event generated by the stimulation unit which activate sensory receptors of a patient, in particular irrespective of the patient's actual perception. As such, stimuli may be perceived by a sense of the patient. Specifically, the stimuli generated by the stimulation unit may be perceived by a tactile sense, in particular by receptors provided in the patient's skin, and/or by a visual sense, in particular by receptors provided in the patient's eyes, and/or by a hearing or auditory sense, in particular by receptors provided in an inner ear of the patient. Upon being sensed by the respective receptors of the patient, these stimuli are then guided from there to a patient's nerve system, thereby causing an activation or stimulation of neurons in the patient's brain or spinal cord.

For example, the stimuli generated by the stimulation unit may be provided as mechanical stimuli, such as tactile or vibratory or vibrotactile stimuli, and/or optical or visual stimuli and/or acoustic stimuli and/or electrical stimuli and/or thermal stimuli and/or olfactory stimuli and/or magnetic stimuli and/or ultrasound stimuli and/or laser stimuli and/or etc. These stimuli may be sensed by corresponding receptors, for example, in the patient's skin or eyes. Specifically, the stimuli generated by the stimulation unit may be tactile stimuli realized or generated by compressed air stimulation. For doing so, the stimulation unit may comprise one or more actuators which is/are coupled to an air pump or compressed air source via a valve which can be selectively opened and closed for generating the stimuli. Alternatively or additionally, the stimuli generated by the stimulation unit may be acoustic stimuli which can be perceived by a hearing or auditory sense of the patient, in particular by receptors provided in an inner ear of the patient. Typically, such stimuli are provided to the inner ear in the form of sound waves through air or bone conduction. Alternatively or additionally, the stimuli generated by the stimulation unit may be magnetic stimuli. Such stimuli may be provided by generating a magnetic field, i.e. an alternating magnetic field. Further, such stimuli may be configured to stimulate peripheral nerves.

The described stimuli, for example the vibrotactile stimuli, may be delivered to fingertips, torso, and forehead of a patient. Alternatively or additionally, they can also be delivered to acupuncture points or trigger points for foot, hand or ear zones reflex massage, as known from reflexology.

The stimulation unit of the suggested treatment device makes use of at least one stimulation channel. That is, the stimulation unit is configured for administering different stimuli to the patient's body via at least one stimulation channel.

In the context of the present disclosure, the term ‘stimulation channel’ refers to a technical channel, i.e. associated to the stimulation unit, and/or to a physiological channel, i.e. associated to the patient's physiology. A technical channel denotes any entity of the stimulation unit which is selectively actuatable for administering stimuli to one or more parts of the patient's body. A physiological channel denotes any anatomic entity of the patient which can be selectively stimulated and which is associated to a neuronal population such that different physiological channels are associated to, at least partly, different neuronal populations. Accordingly, the different stimulation channels may be associated to different anatomic areas of the patient. Alternatively or additionally, the different stimulation channels may be associated to different equivalent rectangular bandwidths (ERB). In general, ERB is a measure used in psychoacoustics which gives an approximation to the bandwidths of the filters in human hearing. In other words, ERB is used for modelling the filters in human hearing as rectangular band-pass filters.

Preferably, the stimulation unit is configured to generate the different stimuli via more than one stimulation channel, i.e. two or more stimulation channels. In this way, a multichannel stimulation may be provided. In such a configuration, each channel may be associated to a different entity of the stimulation unit, i.e. which can be separately and independently actuated to selectively deliver the stimuli to the patient's body. In a state in which the medical device interacts with the patient's body to administer the different stimuli, each stimulation channel may be associated to a different part or anatomical area of the patient's body. Accordingly, when a stimulus is generated via a specific stimulation channel, the thus generated stimulus may be administered to the anatomic area of the patient's body associated to the corresponding stimulation channel.

The stimulation unit may comprise at least one stimulation element, preferably more than one stimulation element, each of which is configured to, upon being actuated, generate stimuli to be administered to the patient's body. Each stimulation element may be separately and selectively actuated. The stimulation elements may be configured and designed to be fastened to the patient. In a state in which the stimulation elements are fastened to the patient, they may be arranged apart from one another and/or may be configured to interact with different parts or anatomical areas of the patient's body. In other words, a first stimulation element of the stimulation unit may be configured to administer stimuli to a first anatomical area and a second stimulation element of the stimulation unit may be configured to administer stimuli to a second anatomical area of the patient's body, wherein the first and the second anatomical area differ from one another, at least partly.

For example, for generating vibratory and/or tactile and/or vibrotactile stimuli, a stimulation element may be provided in the form of an electro-mechanical actuator for converting electrical energy into a movement of a component, such as a rod, which may be configured to mechanically act upon the patient's skin. In such a configuration, the electro-mechanical actuator may be provided in the form of an equal current motor, a linear motor, a voice coil, a piezo-electric transducer or a transformer built up of electro-active polymers which change their shape on the application of an electric current.

Alternatively or additionally, for generating acoustic stimuli, a stimulation element may be provided in the form of a loudspeaker configured to selectively and/or variedly generate tones at a desired frequency and at a desired volume level. According to one configuration, the stimulation unit equipped with such an acoustic stimulation element may comprise or be provided in the form of headphones for the patient.

Alternatively or additionally, for generating visual or optical stimuli, a stimulation element may be provided in the form of or may comprise at least one light source, e.g. in the form of a light-emitting diode, configured to selectively emit light of a desired frequency and at a desired intensity level or brightness. According to one configuration, the stimulation unit may be integrated into glasses configured to be put on by the patient. In this respect, the stimulation unit may comprise stimulation elements which are configured to affect the light guided into the patient's eyes to generate the different visual stimuli. The stimulation elements may be provided in the form of light sources for emitting light and/or shutter and/or filter and/or deflecting elements for affecting the light to be guided into the patient's eyes.

Each stimulation channel may be constituted by or associated to one or more stimulation elements of the stimulation unit. Further, each stimulation element may be associated to one, i.e. only one, stimulation channel or to more than one stimulation channel. When the stimulation unit administers stimuli via a specific stimulation channel, this may mean that the at least one stimulation element associated to the specific stimulation channel is actuated so as to generate and thus administer the stimuli.

For controlling operation of the stimulation unit, the medical device is further equipped with a control unit. As such, the control unit is configured for selectively actuating the stimulation unit, in particular the stimulation elements, so as to administer the different stimuli to the patient's body via the at least one stimulation channel. Specifically, the control unit may be configured to provide electric signals or electric energy which are/is translated or converted by the stimulation unit, in particular the at least one stimulation element, to generate the different stimuli.

Preferably, the control unit is configured to control operation of the stimulation unit such that the stimuli generated by the stimulation unit are configured to suppress or to contribute to the suppression of the pathologically synchronous neuronal activity when being administered to the patient's body. Specifically, the control unit may be configured to set a plurality of stimuli to be administered by the stimulation unit such that at least a part of the plurality of stimuli activates the neuronal population affected by the pathologically synchronous activity when being administered to the patient. In other words, the control unit may be configured to actuate the stimulation unit so as to administer the plurality of stimuli to the patient which, upon being sensed by respective receptors and guided to the patient's nervous system, may cause activation of at least a part of the affected neuronal population.

In dependence on the application of the medical device and the characteristics and timing of the stimuli to be administered, the stimuli generated by the stimulation unit may be configured such that at least a part of the stimuli instantly, i.e. upon administering a first stimulus, suppress or contribute to the suppression of the pathologically synchronous neuronal activity. Alternatively or additionally, the stimuli generated by the stimulation unit may be configured to deploy the intended therapeutic effect, i.e. suppressing the pathologically synchronous neuronal activity, after a part of the stimuli has been administered, in particular a certain time after beginning of the treatment.

As such, the control unit may be configured to control or specify the timing and stimulus characteristics of the different stimuli to be administered to the patient's body via the stimulation channels. For setting the timing of the stimuli, i.e. when the different stimuli are to be generated, the control unit may be configured to specify for each stimulus a stimulus onset, i.e. a point in time at which generation of a single stimulus begins. At a specific stimulus onset, the control unit may be configured to generate only one stimulus or simultaneously more than one stimulus via more than one stimulation channels.

For specifying the stimulus characteristics of the different stimuli, the control unit may be configured to set different parameters for each stimulus. The different parameters may comprise at least one of a stimulus modality, a stimulus frequency, a stimulus intensity or amplitude, in particular a stimulus peak amplitude, a stimulus waveform, a stimulus duration, a stimulus channel and a stimulus envelope.

The parameter ‘stimulus modality’ specifies or is indicative of to the modality or type of stimulus. Accordingly, this parameter may specify whether the stimulus is a tactile stimulus, a vibratory stimulus, a vibrotactile stimulus, an acoustic stimulus, a visual stimulus, an electric stimulus, a thermal stimulus, an olfactory stimuli and/or a magnetic stimuli etc.

The parameter ‘stimulus frequency’ specifies or is indicative of a frequency, in particular a mean frequency, of the stimulus to be generated. For example, when the stimulus is an acoustic stimulus, the stimulus frequency may refer to an acoustic frequency. Specifically, the acoustic frequency may be a tone or pitch frequency. As such, the acoustic stimulus may be constituted by a pure tone and/or by noise, e.g. a narrow band noise. The narrow band noise may have a dominant tone. Thus, the stimulus frequency may define a mean frequency or a frequency of the dominant tone of the acoustic stimulus, in particular of a narrow band noise. Accordingly, when the stimulus is a vibratory stimulus, the stimulus frequency may refer to a vibratory frequency. In case the stimulus is provided in the form of a vibratory burst, the stimulus frequency may be a mean vibratory frequency of the vibratory burst.

The parameter ‘stimulus amplitude’ specifies or is indicative of to an amplitude or intensity of the stimulus, in particular a peak amplitude or intensity.

The parameter ‘stimulus waveform’ defines or is indicative of the waveform of the stimulus to be generated. Accordingly, this parameter may specify whether the stimulus has a sinusoidal or non-sinusoidal waveform and/or whether the stimulus has a sine, triangle or sawtooth waveform.

The parameter ‘stimulus duration’ specifies or is indicative of the duration of the stimulus.

The parameter ‘stimulus channel’ specifies or is indicative of the stimulation channel, in particular the stimulation element, via or by means of which the stimulus is to be generated.

The parameter ‘stimulus envelope’ may specify the course of a stimulus amplitude or intensity over time, in particular during the stimulus duration. For example, the stimulus envelope may specify a mean stimulus amplitude or intensity over time. The stimulus envelope may comprise different phases. For example, the stimulus envelope may have a ramp up phase, i.e. during which the stimulus amplitude rises, a plateau phase, i.e. during which the stimulus amplitude remains constant or substantially constant, and a ramp down phase, i.e. during which the stimulus amplitude decreases. The stimulus envelope may be symmetric, e.g. having correspondingly designed ramp up and ramp down phases. Alternatively the stimulus envelope may be non-symmetric. For example, the stimulus envelope may be symmetric, wherein the ramp up phase and ramp down have a corresponding length and the plateau phase may be scheduled therebetween. In one configuration, the plateau phase may be longer than the ramp up and/or ramp down phase. Such a configuration may be used when longer and steady increases of firing rates are favorable. Non-symmetric stimulus envelopes may be used to induce sharp and tightly controlled neuronal responses that are phase locked to the stimulus, e.g. to a vibratory burst. Specifically, non-symmetric stimulus envelopes may be provided in which a ramp up phase is longer than a ramp down phase. Further, the stimulus envelope may be provided with an overshoot phase, during which the stimulus amplitude rises sharply within the envelope. In this way, phase reset-like brief epochs may be induced. As such, an overshoot may cause brief epochs with particularly pronounced phase locking between the stimulus and neuronal activity and hence may enable precise temporal control of neural discharges.

Alternatively or additionally, the stimulus characteristic may comprise a parameter specifying or being indicative of a psychophysical strength of the stimulus. Specifically, this parameter specifies a subjective level of perception, i.e. how strong a patient perceives the stimulus when being administered. For categorizing the stimulus in terms of psychophysical strength, the psychophysical strength parameter may indicate one of a plurality of equal strength groups. Stimuli which are associated to the same equal strength group are perceived as equally or substantially equally strong by a patient, respectively. For defining equal strength groups and categorizing the different stimuli in terms of psychophysical strength, a psychophysical comparison method may be performed. For example, a method may be used, such as a two-alternative forced choice method or a two-interval forced choice method, in which different stimuli are generated and the patient rates the strength of the different stimuli, in particular by comparing pairwise generated stimuli. Based on these comparisons, the different stimuli may be categorized into different equal strength groups.

The control unit may be configured to set or define a sequence of successive stimuli to be generated by the stimulation unit. Preferably, the control unit sets for each stimulation channel a sequence of stimuli to be generated overtime. In this way, the control unit may be configured to set or provide a stimulation pattern according to which the stimulation unit is actuated. In the context of the present disclosure, the term ‘stimulation pattern’ refers to a control pattern which defines how the stimulation unit, in particular the at least one stimulation element, is to be actuated over time. As such, the stimulation pattern defines timing and characteristics of the stimuli to be generated by the stimulation unit over time. More specifically, the stimulation pattern may specify for each stimulation channel a sequence of actuation intervals over time and for each actuation interval whether a stimulus is to be generated and, if so, its stimulus characteristics.

According to one embodiment, the control unit may be configured to periodically deliver a predefined sequence of stimuli, i.e. which is repeated at regular intervals to form a regular stimulation pattern during and/or among treatments with the medical device. Alternatively, the control unit may deploy a varying stimulation pattern, i.e. which may continuingly vary during and among treatments as described, for example, in WO 2016/207247 A1 and WO 2019/243634 A1. By applying a varying stimulation pattern, the extent and robustness of long-lasting therapeutic and desynchronizing effects may be improved. This may particularly apply when different central oscillations occur in the pathological synchronous activity of a neuronal population, i.e. different neuronal rhythms with different dominant frequencies. For example, this may be achieved by increasing randomness or variation of the stimulation pattern.

For varying the stimulation pattern, the control unit may be configured to vary the sequence over time, for example, by adapting the timing and order of stimuli to be administered, thereby providing randomness of the stimulation pattern in time domain and channel domain, also referred to as spatial domain. For varying the time domain, the stimuli onset of individual stimuli may be variably set, in particular by providing a varying jitter. Further, for varying the channel or spatial domain, characteristics of the stimuli within the stimulation sequences may be variedly set. For example, the stimulus channel parameter may be variedly adapted, thereby variably changing, i.e. in a non-regular manner, the stimulation channel or stimulation element via which the stimulus is to be generated.

The general functional and structural configuration of such a non-invasive medical device for suppressing a pathological synchronous activity of the patient's neurons as well as the approach of generating and implementing stimulation patterns are known to a person skilled in the art, see e.g. WO 2019/243634 A1 and WO 2020/049004 A1, and are therefore not further specified in the present disclosure. Rather, technical features interlinked with the present invention are specified in the following.

As set forth above, the control unit of the proposed medical treatment device is provided for selectively actuating the stimulation unit. Specifically, the control unit is configured to actuate the stimulation unit to successively generate a first stimulus and a subsequent second stimulus in a sequence. In other words, the first stimulus is to be generated prior to the second stimulus and thus precedes the second stimulus in the sequence. Specifically, the control unit may be configured to generate the first and the second stimulus via one, i.e. the same, stimulation channel or via different stimulation channels. In other words, the first stimuli may be generated by a first stimulation channel and the second stimuli may be generated by the first stimulation channel or a second stimulation channel.

Further, the control unit is configured to generate the first and the second stimulus such that a minimum non-stimulation period is provided between the first and the second stimulus. In the context of the present disclosure, the term ‘minimum non-stimulation period’, also referred to as ‘blank window’, refers to a time interval following the first stimulus that should be kept free from the second stimulus, i.e. during which the second stimuli is prevented from being generated. Thus, a pause period between the first and the second stimulus should have at least the duration of the minimum non-stimulation period.

It has been found that, if stimuli generated by the stimulation unit follow too quickly one after the other, i.e. with no proper pause period therebetween, mutual masking and habituation effects may occur which, in turn, significantly reduce therapeutic stimulation effects. In other words, if stimuli generated by the stimulation unit follow too quickly one after the other, the intended therapeutic effect of suppressing abnormally synchronized neuronal activity may not or only insufficiently be achieved. This applies to both closely timed stimuli generated via the same stimulation channels causing intra-channel masking and habituation effects and via different stimulation channels causing cross-channel masking and habituation effects. Accordingly, by ensuring that the minimum non-stimulation period is provided between successive stimuli generated via the same or different stimulation channels, the proposed device may effectively prevent mutual masking and habituation effects during treatment. As a result, the proposed medical device may contribute to effective treatment procedures.

Specifically, when the first and the second stimulus are generated via one, i.e. the same stimulation channel, the non-stimulation period describes a time period associated to the stimulation channel during which no stimulus is to be generated via that stimulation channel. In other words, the stimulation channel is to be kept stimulus-free during the non-stimulation period. As such, the control unit may be configured to, when the first and the second stimulus are generated via one stimulation channel, provide a pause period between the first and the second stimulus which has at least the length of the minimum non-stimulation period and during which the stimulation channel is kept stimulus-free. When the first and the second stimulus are generated via different stimulation channels, the minimum non-stimulation period describes a time period during which no stimulus is to be generated by the different stimulation channels or by any other stimulation channel associated to the stimulation unit. Thus, when the first and the second stimulus are generated via different stimulation channels, the control unit may be configured to provide a pause period between the first and the second stimulus which has at least the length of the minimum non-stimulation period and during which the different stimulation channels or all stimulation channels of the stimulation unit are kept stimulus-free. Accordingly, the control unit may be configured to prevent the stimulation unit from generating stimuli via the at least one stimulation channel used for generating the first and the second stimulus during a time period provided between the first stimulus and the second stimulus.

In a further development, the control unit may be configured to subsequently generate more than two stimuli in a sequence via one or more stimulation channels. In such a stimuli sequence, each stimulus is to be generated a corresponding minimum non-stimulation period after a preceding stimulus in the sequence generated or to be generated via the same stimulation channel as the stimulus and/or via another stimulation channel.

Further, for ensuring that the non-actuation periods are properly set, the control unit of the proposed medical device is configured to set the duration of the minimum non-stimulation period in dependence on at least one stimulus characteristic of the first stimulus. It has been further found that the duration of a proper minimum non-stimulation period between two successive stimuli generated via the same or different stimulation channel substantially depends on the stimulus characteristic of the preceding of the two successive stimuli. Accordingly, by providing the minimum non-stimulating periods between successive stimuli and taking into account the stimulus characteristic of the preceding of the two successive stimuli when defining the duration of the minimum non-stimulation period, the proposed medical device may allow for effective treatment of neurological and psychiatric disorders.

In a further development, the control unit may be configured to set the duration of the minimum non-stimulation period in dependence on at least one stimulus characteristic of the second stimulus in addition to the at least one stimulus characteristic of the first stimulus.

Each stimulus when being administered to the patient's body is configured to stimulate a target neuronal population. It has been found that the closer the target neuronal populations stimulated by two successive stimuli are located, the longer the minimum non-stimulation period between these two stimuli is to be set in order to effectively avoid mutual masking and habituation effects. By such measures, it may be avoided that stimuli, which may stimulate overlapping or neighboring neuronal populations, are generated too quickly one after the other, thereby preventing masking and habituation effects.

Thus, when determining the duration of the minimum non-stimulation period, the control unit may be configured to take into account the target neuronal population to be stimulated by the first and the second stimulus. In other words, the control unit may be configured to set the duration of the minimum non-stimulation period between the first and the second stimulus in dependence on the target neuronal populations to be stimulated by the first and the second stimulus.

The stimulus characteristics of the first and the second stimuli may be indicative of the target neuronal populations to be stimulated by the respective stimulus. Accordingly, by taking into account the stimulus characteristics of the first and the second stimulus when determining the duration of the minimum non-stimulation period, masking and habituation effects may be effectively. Accordingly, these stimulus characteristics are preferably indicative of the target neuronal population to be stimulated by the respective stimulus. Specifically, such stimulus characteristics may comprise at least one of a stimulus modality, a stimulus frequency, a stimulus amplitude, a stimulus waveform, a stimulus duration, and stimulus channel.

More specifically, the control unit may be configured to set the duration of the minimum non-stimulation period in dependence on a stimulus duration of the first stimulus. Additionally, the control unit may be configured to take into account the stimulus characteristics of the second stimulus when setting the duration of the minimum non-stimulation period. Specifically, the control unit may be configured to set the duration of the minimum non-stimulation period in dependence on a stimulus channel of both the first and the second stimulus and/or a stimulus frequency of both the first and the second stimulus.

According to one configuration, the control unit may be configured to determine whether the successive first and second stimulus are generated via one stimulation channel, i.e. the same stimulation channel, or via different stimulation channels. In response thereto, the control unit may be configured to set the duration of the minimum non-stimulation period in the range of 1.5 to 6 times or 2 to 4 times, for example 3 or substantially 3 times, the duration of the first stimulus when the first and the second stimulus are generated via different stimulation channels, in particular when the first and the second stimulus are generated via neighboring or directly neighboring stimulus channels. Alternatively or additionally, the control unit may be configured to set the duration of the minimum non-stimulation period in the range of 25% to 60%, for example 40% or substantially 40% of the stimulus duration of the first stimulus when the first and the second stimulus are generated via different stimulation channels, in particular when they are generated by stimulations channels which are not neighboring, i.e. which are located further apart than neighboring stimulation channels. Alternatively or additionally, the control unit may be configured to set the duration of the minimum non-stimulation period in the range of 2.5 to 6 times or 3 to 5 times, for example 5 times or substantially 5 times, the stimulus duration of the first stimulus when the first and the second stimulus are generated via one stimulation channel, i.e. the same stimulation channel.

Additionally, the control unit may be configured to set the duration of the minimum non-stimulation period in the range of 2.5 to 6 times, for example 5 times, the stimulus duration of the first stimulus when the first and the second stimulus are generated via one stimulation channel, i.e. the same stimulation channel, and a stimulus frequency of the first and the second stimulus coincide or substantially coincide. Further, the control unit may be configured to set the duration of the minimum non-stimulation period less than 3 times the stimulus duration of the first stimulus when the first and the second stimulus are generated via one stimulation channel, i.e. the same stimulation channel, and a stimulus frequency of the first stimulus differs from a stimulus frequency of the second stimulus. In other words, the control unit may be configured to set the duration of the minimum non-stimulation period in dependence on the stimulus frequency of the first and the second stimulus, in particular such that the duration of the minimum non-stimulation period is set to be longer when the stimulus frequency of the first and the second stimulus coincide or substantially coincide compared to a configuration in which the stimulus frequency of the first and the second stimulus differ.

As set forth above, the control unit of the medical device is configured to keep the defined minimum non-stimulation periods stimulus-free for the corresponding stimulation channels. That means that the control unit constraints operation of the stimulation unit such that the stimulation unit is not allowed to generate the second stimulus via the corresponding stimulation channel for the duration of the minimum non-stimulation period after the first stimulus. In other words, the control unit may be provided so as to comply with a non-stimulation criterion requiring the stimulation unit to deliver stimuli via a stimulation channel only when they are administered a corresponding minimum non-stimulation period after a preceding stimulus, respectively. In general, a stimulus falling within a non-stimulation period may be denoted as ‘non-stimulation period violation’ or ‘blank window violation’.

For avoiding or preventing such non-stimulation period violations, i.e. to enable that the minimum non-stimulation period is provided between the first and the second stimulus, the control unit may be configured to apply stimulus adaptation techniques as exemplarily described in the following.

For example, for avoiding non-stimulation period violations, the control unit may take measures to shorten the minimum non-stimulation period. For doing so, the control unit may be configured to adapt the characteristic of the preceding stimulus, i.e. the first stimulus, since the duration of the non-stimulation period depends there onto, in particular on the stimulus duration and stimulus frequency, e.g. vibratory and/or acoustic frequency, of the preceding stimulus. Specifically, the control unit may adapt the characteristic of the preceding stimulus such that a stimulus duration is decreased or shortened, in particular while the stimulus frequency and psychophysical strength remain unchanged. In order to maintain the psychophysical strength unchanged, the stimulus amplitude of the first stimulus, at the same time, may be increased. This is due to the fact that a patient may perceive a first stimulus and a second stimulus as equally strong when the second stimulus has a longer stimulus duration but lower stimulus amplitude, or vice versa, compared to the first stimulus.

Alternatively or additionally, for avoiding non-stimulation period violations, the control unit may be configured to adapt the characteristic of the first or the second stimulus by adapting, in particular its stimulus frequency, e.g. the vibratory and/or acoustic frequency, such that the stimulus frequency of the first and second stimulus differ, in particular qualitatively differ, from one another, in particular when the first and the second stimulus are to be generated via the same stimulation channel. In this way, the first stimulus and the second stimulus may stimulate, at least partly, different neuronal populations while being delivered via the same stimulation channel, thereby avoiding or counteracting the occurrence of mutual masking and habituation effects.

Alternatively or additionally, for avoiding non-stimulation period violations, the control unit may be configured to adapt the characteristic of the first or the second stimulus by adapting its stimulus channel such that the stimulation channel via which the first and the second stimulus are to be delivered differ from one another. Accordingly, the first stimulus and the second stimulus may stimulate, at least partly, different neuronal populations by being generated via different stimulation channels, thereby avoiding or counteracting the occurrence of mutual masking and habituation effects.

In a further development, the control unit may be configured to generate the first and the second stimulus via a first stimulation channel and to generate a third stimulus via a second stimulation channel, wherein the third stimulus is to be generated prior to the second stimulus such that a further minimum non-stimulation period is provided between the third and the second stimulus. Further, the control unit may be configured to set the duration of the further minimum non-stimulation period in dependence on at least one stimulus characteristic of the third stimulus.

Specifically, the control unit may be configured to set the duration of the further minimum non-stimulation period in dependence on a stimulus channel of both the second and the third stimulus.

As set forth above, the control unit may be configured to set or provide a stimulation pattern according to which the stimulation unit is actuated, i.e. which defines how the stimulation unit, in particular the at least one stimulation element, is to be actuated over time. In a further development, the control unit may be configured to take into account stimulation channel specific propagation delays when actuating stimulation units, in particular when setting or providing the stimulation pattern. As to substance, it has been found that a propagation delay of stimuli may differ in dependence on the characteristics of a stimulation channel, e.g., may differ among vibrotactile stimulators, tone frequencies of acoustic stimuli, optical stimuli, etc. In general, the propagation delay can be assessed as time between stimulus onset and peak of the (averaged) evoked response, which can be measured with, e.g., electroencephalography, magnetoencephalography or other electrophysiological means, such as epicortical or epidural electrodes. Alternatively, instead of the averaged evoked response one can use the time of peak phase reset, where the phase is calculated for the relevant, pathological brain rhythm. For taking into account the propagation delays of the different stimulation channels, the control unit may associate to each stimulation channel a channel specific propagation delay. This propagation delay may be used to define when actuation of the stimulation unit, in particular the stimulation elements, is to be performed to induce a desired stimulus effect at a desired time period.

### BRIEF DESCRIPTION OF THE DRAWINGS

The present disclosure will be more readily appreciated by reference to the following detailed description when being considered in connection with the accompanying drawings in which:

FIG. 1 is a schematic view of a medical device for stimulating neurons of a patient to suppress a pathologically synchronous activity thereof;

FIG. 2 illustrates a stimulation pattern according to which the medical device depicted in FIG. 1 is operated;

FIG. 3 illustrates a further stimulation pattern according to which the medical device depicted in FIG. 1 is operated;

FIG. 4 illustrates a procedure deployed by a control unit of the device depicted in FIG. 1 for generating a stimulation pattern which;

FIGS. 5 and 6 depict parts of a stimulation pattern for illustrating individual steps of the procedure depicted in FIG. 4;

FIGS. 7a-7c depict different forms of stimulus envelopes;

FIGS. 8 and 9 depict parts of a stimulation pattern for illustrating individual steps of the procedure depicted in FIG. 4; and

FIG. 10 is a schematic view of a medical device according to a further embodiment.

### DETAILED DESCRIPTION OF PREFERRED EMBODIMENTS

In the following, the invention will be explained in more detail with reference to the accompanying Figures. In the Figures, like elements are denoted by identical reference numerals and repeated description thereof may be omitted in order to avoid redundancies.

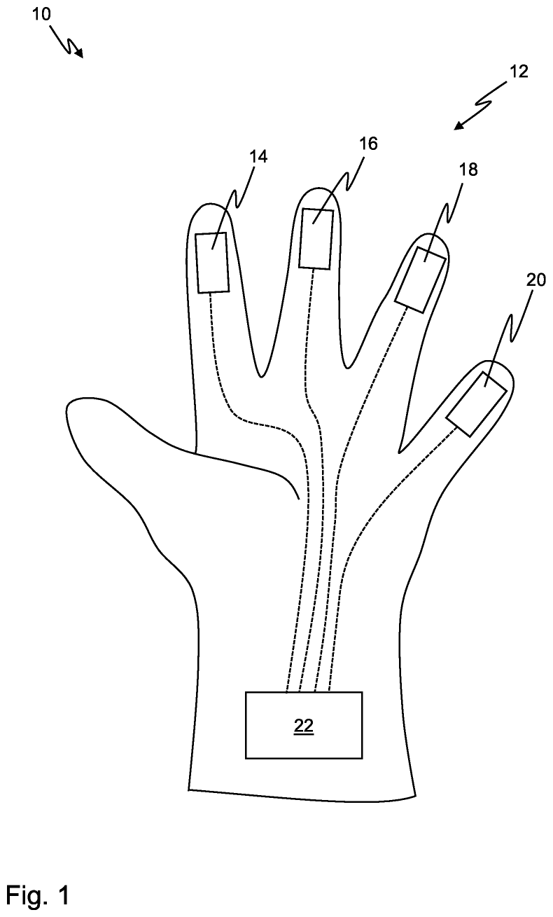

FIG. 1 depicts a medical device 10 for stimulating neurons of a patient which is used to treat a pathologically synchronous activity thereof. In other words, the medical device 10 is intended to be used for the treatment of neurological or psychiatric disorders or diseases caused by a pathologically synchronous neuronal activity, such as Parkinson's disease. The medical device 10 is provided in the form of a therapeutic glove, a bottom side, i.e. glove palm, of which is depicted in FIG. 1. In the shown configuration, the glove is configured to be put on a patient's hand for acting upon distal phalanges of the patient's fingers.

The medical device 10 is a non-invasive therapeutic device which, for acting upon the patient's body, comprises a non-invasive stimulation unit 12 configured for administering different stimuli to the patient's body via a plurality of stimulation channels 14-20. Specifically, each stimulation channel is formed by a stimulation element 14-20 configured to administer stimuli to a surface of the patient's hand. The stimulation elements 14-20 are separately and selectively actuatable to generate the different stimuli, wherein each stimulation channel 14-20 is associated to at least one stimulation element 14-20. The stimulation elements 14-20 are configured to, in a state in which they are attached to or interact with the patient's body, deliver stimuli to different anatomical areas of the patient's body. Specifically, the stimulation elements 14-20 are provided such that, in a state in which the medical device 10 is fastened to the patient's hand, each stimulation element 14-20 is arranged at a respective finger tip of the patient's hand, in particular at the phalanges of the respective fingers. In an alternative embodiment, the medical device 10 may be configured to be fastened to other parts of the patient's body, preferably such that the different stimulating elements 14-20 can be fastened to different sites of the patient's body.

In the shown configuration, the stimulation elements 14-20 are configured to provide mechanical stimuli, e.g. tactile and/or vibratory or vibrotactile stimuli. In an alternative configuration, the stimulation unit may be configured to alternatively or additionally administer at least one of visual stimuli, electrical stimuli and acoustic stimuli. Specifically, for providing mechanical stimuli, the stimulation elements 14-20 may comprise an electro-mechanical actuator for converting electrical energy into a movement of a rod element to mechanically act upon the patient's skin. For providing electrical energy to the electro-mechanical actuator, the stimulation unit 12 may comprise or be connected to an energy source, e.g. in the form of a battery. By such a configuration, the stimulation elements 14-20 may be variably driven so as to generate qualitatively different stimuli, i.e. of varying stimulus frequencies, amplitudes, durations, waveforms etc.

In general, the human skin comprises mechanoreceptive afferent units capable of sensing stimuli, i.e. tactile or vibratory stimuli, which have been classified into two major categories, namely into fast-adapting units (FA) and slow-adapting units (SA). The FA units respond to moving stimuli as well as the onset and removal of a step stimulus. In contrast, the SA units respond with a sustained discharge. In addition, based on the properties of their receptive fields, both categories are further classified into two different types. The fast-adapting type I (FA I) units, also referred to as RA (rapidly adapting) units, and the slow-adapting type I (SA I) units form a small, but clearly delimited receptive fields on the surface of the skin. In contrast, the receptive fields formed by the fast-adapting type II (FA II) units, also referred to as PC (Pacinian corpuscles) units, and the slow-adapting type II (SA II) are wider and have obscure borders.

Typically, the distribution and density of the different types of mechanoreceptors differ in dependence on the position on the human skin. For example, regarding the glabrous skin of the human hand, the density of FA I units is relatively high in an area of the fingertips, i.e. the FA I units are predominantly located in the fingertips. In contrast, the spatial density of FA II units is more uniformly distributed across the entire hand, but still largest in the fingertips.

The four different types of human cutaneous mechanoreceptors respond optimally to qualitatively different stimuli. Specifically, edge stimuli and stretch stimuli are optimal for SA I and SA II mechanoreceptors, respectively. SA I units often have a rather irregular sustained discharge, whereas SA II units discharge in a regular manner, but often display spontaneous discharge in the absence of tactile stimulation. Vibratory perpendicular sinusoidal skin displacements in the range between about 30 Hz to about 60 Hz are optimal stimuli for FA I units, whereas vibratory stimuli in the range between about 100 Hz to about 300 Hz are optimal stimuli for FA II units. FA I and, especially, SA I units have a pronounced edge contour sensitivity and, hence, their response is stronger when a stimulating contactor surface which is not completely contained in the receptive field. Accordingly, to enhance the FA I responses, instead of a flat, spatially homogenous contactor surface of the stimulation element one could use a contactor surface with a spatially inhomogeneous indentation profile.

In the shown embodiment, the stimulation elements 14-20 may be designed and configured to generate stimuli adapted to the response characteristic of FA I, FA II, SA I and/or SA II units. For example, the stimulation elements 14-20 may generate stimuli which are adapted to the response characteristic of one of the FA I, FA II, SA I and SA II units. Alternatively or additionally, the stimulation elements 14-20 may be configured to generate stimuli targeting more than one of the FA I, FA II, SA I and SA II units. Alternatively or additionally, the stimulation elements 14-20 may be configured for being operated in different operational modes in which different stimuli are generated which, respectively, are adapted to a response characteristic of different FA I, FA II, SA I and SA II units.

Specifically, for targeting FA I type receptors, the stimulation elements 14-20 may be configured to generate vibratory stimuli with a vibration frequency between 30 Hz to 60 Hz, i.e. 30 Hz, and a vibration peak-to-peak amplitude of 0.25 mm. By such a configuration, the stimulation elements 14-20 are adapted to the stimulation of fingertips of the patient's hand. Alternatively, for targeting FA II type receptors, a stimulation element may be configured to generate vibratory stimuli with a vibratory frequency between 100 Hz to 300 Hz, i.e. 250 Hz, and a peak-to-peak amplitude of 2.0 mm. For this configuration, a greater vibration frequency may be used, in particular to provide an impression of equal loudness.

For controlling operation of the stimulation unit 12, the medical device 10 is further equipped with a control unit 22. The control unit 22 is configured for selectively and intermittently actuating the stimulation unit 12, in particular the different stimulation elements 14-20. Specifically, for achieving the intended therapeutic effect, the control unit 22 is configured to actuate the stimulation unit 12 to generate a sequence of successive stimuli via the different stimulation channels 14-20. For doing so, the control unit 22 is configured to provide a stimulation pattern, i.e. a control pattern, according to which the different stimulation elements 14-20 are actuated over time. As such, the stimulation pattern specifies for each stimulation channel 14-20 a sequence of stimuli to be generated in which actuation periods and pause periods alternate.

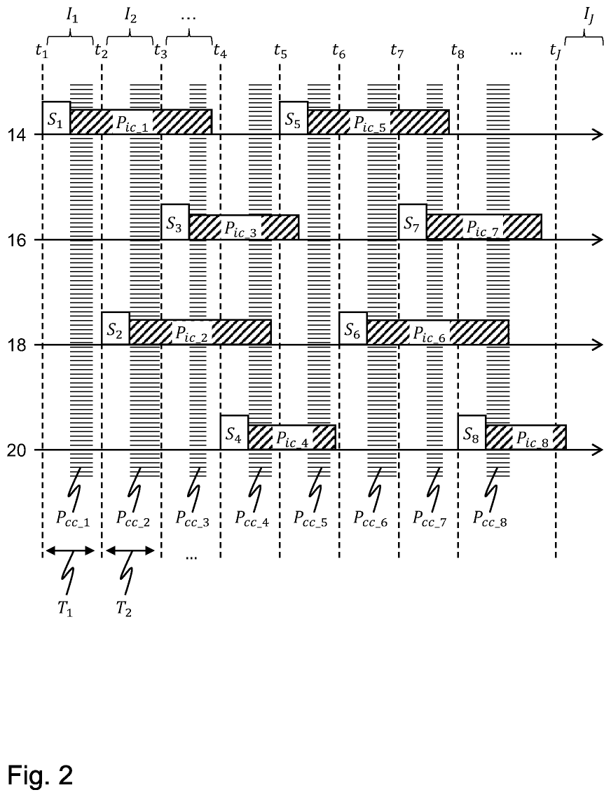

FIG. 2 depicts an exemplary multichannel stimulation pattern generated by the control unit 22. The stimulation pattern is constituted by successive actuation intervals I_{1, . . . J}, wherein J denotes a total number of actuation intervals comprised in the sequence of stimuli to be generated. Within each actuation interval Iⱼ, one stimulus Sⱼ is generated so as to provide the sequence of stimuli, wherein j denotes a sequence number which may assume a value between 1 and J.

The shown stimulation pattern is a regular stimulation pattern, i.e. which is provided with minimum randomness in time domain and in the channel domain. This means that the actuation intervals are provided at regular time intervals and the actuation order or pattern of the different stimulation channels 14-20 is maintained throughout the stimulation sequence. Specifically, in the shown configuration, the actuation order of the stimulation channels is: channel 14-channel 18-channel 16-channel 20. This actuation order is constantly repeated throughout the sequence.

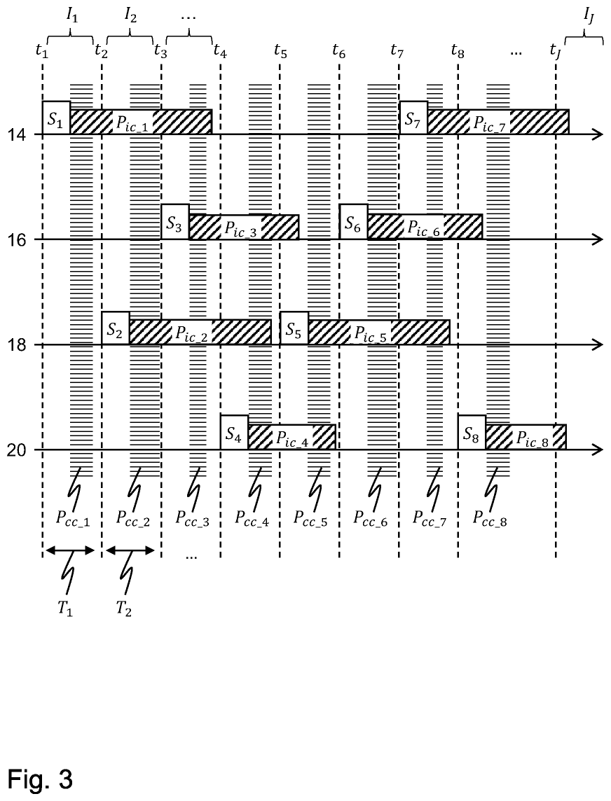

The control unit 22 is not limited to the generation of regular stimulation pattern. Rather, the control unit 22 is capable and configured to further generate stimulation pattern with varying, non-regular actuation orders may be provided as depicted, for example, in FIG. 3, thereby increasing randomness in the channel domain, also referred to as spatial domain, of the stimulation pattern. Alternatively or additionally, the control unit 22 may be configured to generate stimulation patterns in which the length of actuation intervals is varied within and among sequences, thereby increasing randomness in the time domain.

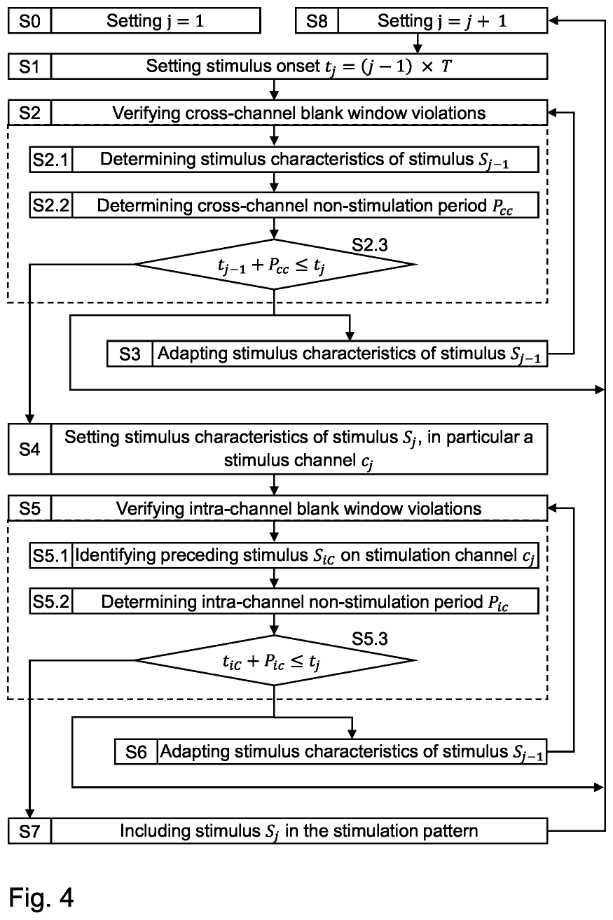

In the following, with reference to FIG. 4, a generic procedure carried out by the control unit 22 is described for generating a stimulation pattern, for example, as depicted in FIGS. 2 and 3. In the procedure, the different actuation intervals Iⱼ constituting the stimulation sequence are defined successively, i.e. one after the other. Accordingly, the process starts with setting the parameter j to 1 in step S0 and thereafter iteratively passes through steps S1-S7, wherein in each iteration the timing and characteristics of the stimulus Sⱼ to be administered during one actuation interval, i.e. the j^{th} actuation interval Iⱼ, is defined.

Specifically, the stimulation pattern to be generated may be a periodic stimulation pattern, i.e. in which stimulations are intermittently provided and triggered at regular time intervals as, for example, illustrated in FIG. 2. This means that actuation of the different stimulation channels 14-20 may be triggered according to a periodic time sequence, also referred to as beat, given by:

$${t}_{j} = ( j - 1 ) \times T , \qquad ( 1 )$$

wherein tⱼ denotes a stimulus onset, i.e. the point in time at which delivery of a stimulus Sⱼ begins, and T denotes a constant time interval between successive time onsets which may be expressed as:

$$T = {t}_{j} - {t}_{j - 1} \qquad ( 2 )$$

As such, each stimulus onset tⱼ is associated to an actuation interval Iⱼ. Accordingly, in step S1, a stimulus onset tⱼ for the associated j^{th} actuation interval Iⱼ is set. In this way, a regular pattern having minimal randomness in the time domain may be achieved. Alternatively or additionally, as already mentioned above, the control unit 22 may be configured to vary the length of the time interval between successive stimulus onsets within the stimulation sequence, e.g., by employing an exponential distribution process and/or a Markov process and/or any other suitable stochastic or deterministic or combined stochastic-deterministic process. According to one approach, the control unit 22 may be configured to variedly provide a deviation, also referred to as jitter, from regular onsets, e.g., as defined in above equations (1) and (2).

In a next step S2, the control unit 22 verifies whether a proper pause period is provided between a preceding stimulus Sⱼ₋₁ and the stimulus Sⱼ and takes appropriate measures if this is not the case. In this context, the ‘preceding stimulus Sⱼ₋₁’ refers to a stimulus to be generated by any one of the different stimulation channels 14-20 and which precedes, i.e. directly precedes the stimulus Sⱼ in the stimulation sequence. Further, the proper pause period is also referred to as minimum cross-channel non-stimulation period P_{cc} and refers to a time interval following preceding stimulus Sⱼ₋₁ that should be kept free from stimulus Sⱼ, wherein the stimulus Sⱼ and the preceding stimulus Sⱼ₋₁ may be generated via the same or different stimulation channels 14-20. This step therefore is referred to as ‘verifying cross-channel blank window violations’.

For doing so, in a first sub-step S2.1, the stimulus characteristics of the preceding stimulus Sⱼ₋₁ are determined. Then, based on the thus determined characteristics, the control unit 22 sets the duration of the minimum cross-channel non-stimulation period P_{cc} in sub-step S2.2. Specifically, the control unit 22 may be configured to set the minimum cross-channel non-stimulation period P_{cc} in dependence on at least one of a stimulus channel, stimulus modality, stimulus duration, stimulus amplitude, stimulus waveform, stimulus frequency or a psychophysical strength of the preceding stimulus Sⱼ₋₁. For example, the longer the stimulus duration of the preceding stimulus Sⱼ₋₁ the longer the minimum cross-channel non-stimulation period P_{cc} may be set.

Thereafter, in sub-step S2.3, the control unit 22 determines whether the minimum cross-channel non-stimulation period P_{cc} is provided between the stimulus Sⱼ and the preceding stimulus Sⱼ₋₁ and thus whether the cross-channel minimum non-stimulation criterion is met. If this is true, the procedure proceeds to step S4.

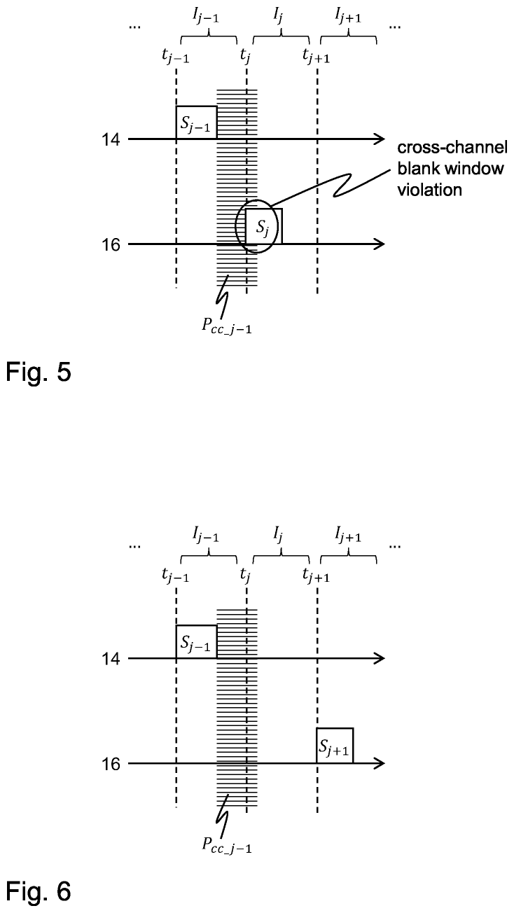

However, if between the stimulus Sⱼ and the preceding stimulus Sⱼ₋₁ the minimum non-stimulation criterion is not met, i.e. the stimulus Sⱼ falls within the cross-channel non-stimulation period P_{cc} following the preceding stimulus Sⱼ₋₁ as exemplary depicted in FIG. 5, then appropriate measures may be taken by the control unit 22 as will be described in the following.

As can be gathered from FIG. 5, a cross-channel blank window violation occurs when the stimulus onset tⱼ of stimulus Sⱼ lies within the minimum cross-channel non-stimulation period P_{cc_j-1}. In this case, the procedure may proceed from step S2.3 to Step S8 in which the sequence number j is increased by 1 before running through a subsequent iteration by returning to step S1. By doing so, stimulation in the actuation interval Iⱼ may be omitted, i.e. no stimulation is performed in this actuation interval. The result of this approach is depicted in FIG. 6.

Alternatively, when a cross-channel blank window violation is determined in step S2.3, the method may proceed to step S3 in which the stimulus characteristics of the preceding stimulus Sⱼ₋₁ are adapted, for example, by shortening the stimulation duration and/or by changing the stimulation frequency of the preceding stimulus Sⱼ₋₁. These measures take into account that the duration of the minimum cross-channel non-stimulation period P_{cc} depends on the stimulus characteristics of the preceding stimulus Sⱼ₋₁. That is, by shortening the stimulation of the preceding stimulus Sⱼ₋₁ and/or by changing the stimulation frequency, the duration of the minimum cross-channel non-stimulation period P_{cc} may be shortened, thereby allowing the stimulus onset tⱼ to no longer fall within the minimum cross-channel non-stimulation period P_{cc}.

As a result, the control unit 22 is configured to actuate the stimulation unit 12 to successively generate the stimulus Sⱼ and its preceding stimulus Sⱼ₋₁ in a sequence such that a minimum cross-channel non-stimulation period P_{cc} is provided between the stimulus Sⱼ and its preceding stimulus Sⱼ₋₁. Further, the control unit 22 is configured to prevent the stimulation unit 12 from generating stimuli via the one or more stimulation channel used for generating the stimulus Sⱼ and the preceding stimulus Sⱼ₋₁ during a period provided between the stimulus Sⱼ and the preceding stimulus Sⱼ₋₁.

In step S4, the stimulus characteristics of the stimulus Sⱼ is set. Specifically, the control unit 22 specifies a stimulus modality mⱼ, stimulus channel cⱼ, stimulus frequency vⱼ, stimulus duration dⱼ, stimulus amplitude aⱼ, stimulus waveform wⱼ and equal strength parameter eⱼ. Accordingly, the stimulus to be administered may be expressed by the following vector parameter:

$${S}_{j} = [ {m}_{j} ; {c}_{j} ; {ν}_{j} ; {d}_{j} ; {a}_{j} ; {w}_{j} ; {e}_{j} ] \qquad ( 3 )$$

Specifically, the stimulus modality mⱼ denotes the type of stimulus, e.g. whether the stimulus is a tactile stimulus, a vibratory stimulus, a vibrotactile stimulus, a visual stimulus, an electrical stimulus and an acoustic stimulus. As described above, in the shown configuration of the medical device 10, the stimulation unit 12 is configured to provide vibrotactile stimuli such that all stimuli in the sequence to be generated are of this stimulus modality.

The parameter stimulus channel cⱼ denotes the stimulation channel, i.e. the first to fourth stimulation channel 14-20, via which the stimulus Sⱼ is to be generated and accordingly may be expressed as:

$${c}_{j} = \{ \begin{matrix}1 : first stimulation channel 14 \\ 2 : second stimulation channel 16 \\ 3 : third stimulation channel 18 \\ 4 : fourth stimulation channel 20\end{matrix} \qquad ( 4 )$$

In other words, the parameter cⱼ may assume a value between 1 to 4, wherein each value is associated to another stimulation channel 14-20. In the shown configuration, for illustrative purposes, the number of different stimulation channels is exemplary set to four. Of course, the number of different stimulation channels is not confined to four.

The parameter stimulus frequency vⱼ denotes a stimulus frequency, i.e. a mean frequency of the stimulus. The parameter stimulus duration dⱼ denotes the duration of the stimulus Sⱼ to be generated, i.e. the actuation time period. The parameter stimulus amplitude aⱼ denotes the peak amplitude of the stimulus Sⱼ to be generated. The parameter stimulus waveform wⱼ denotes how the stimulation amplitude changes over time, e.g. in a sinusoidal or non-sinusoidal form. Specifically, the stimulus waveform wⱼ may be expressed as:

$${w}_{j} = \{ \begin{matrix}1 : sine wave \\ 2 : triangle wave \\ 3 : sawtooth wave\end{matrix} \qquad ( 5 )$$

and accordingly may assume a value between 1 to 3, wherein each value is associated to another waveform.

Further, the equal strength parameter eⱼ is indicative of the psychophysical strength and may indicate one of a plurality of equal strength groups, wherein stimuli associated to the same equal strength group are perceived as equally or substantially equally strong by a patient. The equal strength parameter eⱼ may be determined as a function of the stimulus frequency vⱼ and the stimulus duration dⱼ.

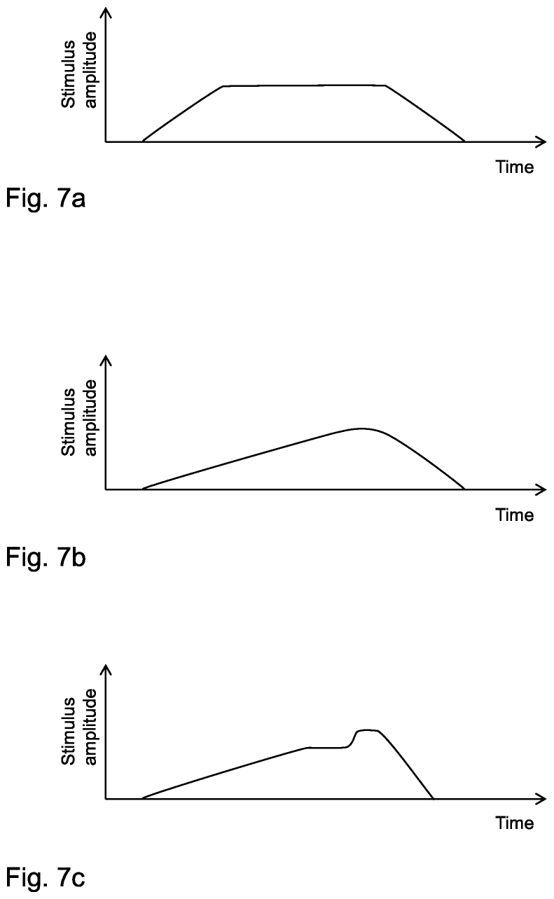

Further, when setting the stimulus characteristics, the control unit 22 may specify a stimulus envelope, e.g. when the stimulus is provided in the form of a vibratory burst. For example, a symmetric stimulus envelope may be provided as depicted in FIG. 7A. In this configuration, the stimulus envelope may have a relatively short ramp up and ramp down phases and a relatively long plateau phase. Alternatively, a non-symmetric stimulus envelope may be provided as depicted in FIG. 7B. In this configuration, the stimulus envelope may have a relatively long ramp up phase and a relatively short ramp down phase. Alternatively, a stimulus envelope may be provided as depicted in FIG. 7C which has an overshoot, i.e. a sudden rise during the plateau phase.

Then, in step S5, the control unit 22 verifies whether a proper pause period is provided between the stimulus Sⱼ and an intra-channel preceding stimulus S_{ic}. In this context, the ‘intra-channel preceding stimulus’ refers to a stimulus to be generated via the same stimulation channel cⱼ as the stimulus Sⱼ and which precedes, in particular directly precedes, the stimulus Sⱼ in the sequence of a common stimulation channel cⱼ. Further, in this respect, the proper pause period is also referred to as ‘minimum intra-channel non-stimulation period P_{ic}’ and refers to a time interval following the intra-channel preceding stimulus S_{ic} that should be kept free from stimulus Sⱼ, wherein the stimulus Sⱼ and the intra-channel preceding stimulus S_{ic} are to be generated via the same stimulation channel 14-20. This step therefore is referred to as ‘verifying intra-channel blank window violations’.

For doing so, in a first sub-step S5.1, the control unit 22 identifies an intra-channel preceding stimulus S_{ic} which is to be generated prior to the stimulus Sⱼ via the same stimulation channel cⱼ. Then, in sub-step S5.2, the control unit 22 determines the duration of the minimum intra-channel non-stimulation period P_{ic} in dependence on stimulus characteristics of the intra-channel preceding stimulus S_{ic}. Specifically, the control unit 22 may be configured to set the minimum intra-channel non-stimulation period P_{ic} in dependence on at least one of a stimulus duration d_{ic} and a stimulus frequency v_{ic} of the intra-channel preceding stimulus S_{ic}. Additionally, for setting the duration of the minimum intra-channel non-stimulation period P_{ic}, the control unit 22 may be configured to further take into account the stimulus characteristics of the stimulus Sⱼ, in particular its stimulus frequency vⱼ. This may be performed such that the longer the stimulus duration d_{ic} of the intra-channel preceding stimulus S_{ic} the longer the minimum intra-channel non-stimulation period P_{ic} is set. Further, in case the stimulation frequencies of the intra-channel preceding stimulus S_{ic} and the stimulus Sⱼ are equal or substantially equal, the duration of the minimum intra-channel non-stimulation period is set to be longer compared to a sequence in which the stimulation frequencies differ among the two stimuli S_{ic}, Sⱼ.

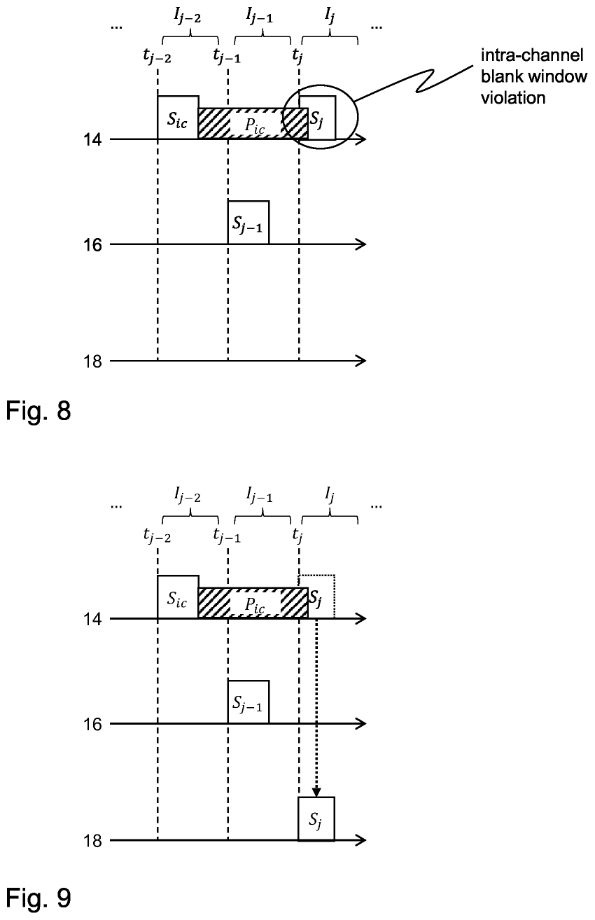

Thereafter, in sub-step S5.3, the control unit 22 determines whether the minimum intra-channel non-stimulation period P_{ic} is provided between the stimulus Sⱼ and the intra-channel preceding stimulus S_{ic} and thus whether the intra-channel minimum non-stimulation criterion is met. If this is true, the process proceeds to step S7. However, if between the stimulus Sⱼ and the intra-channel preceding stimulus S_{ic} the minimum non-stimulation criterion is not met, i.e. the stimulus Sⱼ falls within the intra-channel non-stimulation period P_{ic} following the intra-channel preceding stimulus S_{ic} as exemplary depicted in FIG. 8, then appropriate measures may be taken by the control unit 22 as will be described in the following.

As can be gathered from FIG. 8, an intra-channel blank window violation occurs when the stimulus onset tⱼ of stimulus Sⱼ lies within the minimum intra-channel non-stimulation period P_{ic_j-1}. In this case, the procedure may proceed from step S2.3 to step S8 in which the sequence number j is increased by 1 before running through a subsequent iteration by returning to step S1. By doing so, stimulation in the actuation interval Iⱼ may be omitted, i.e. no stimulation is performed in this actuation interval.

Alternatively, when an intra-channel blank window violation is determined in step S5.3, the method may proceed to step S6 in which the stimulus characteristics of the intra-channel preceding stimulus S_{ic} are adapted, for example, by shortening the stimulation duration and/or by changing the stimulation frequency of the intra-channel preceding stimulus S_{ic}. These measures take into account that the duration of the minimum intra-channel non-stimulation period P_{ic} depends on the stimulus characteristics of the intra-channel preceding stimulus S_{ic}. That is, by shortening the stimulation of the intra-channel preceding stimulus S_{ic} and/or by changing the stimulation frequency, the duration of the minimum intra-channel non-stimulation period P_{ic} may be shorten, thereby allowing that the stimulus onset tⱼ no longer falls within the minimum intra-channel non-stimulation period P_{ic}. Preferably, this step may be performed so as to maintain the equal strength characteristic of the intra-channel preceding stimulus S_{ic}. In other words, the intra-channel preceding stimulus S_{ic} after and before the adaptation performed in step S6 is preferably categorized in the same equal strength group.

Alternatively, when an intra-channel blank window violation is determined in step S5.3, the control unit 22 may be configured to adapt the stimulus channel of the stimulus Sⱼ or the intra-channel preceding stimulus S_{ic} such that their stimulation channels differ as exemplary depicted in FIG. 9 by an arrow.

As a result, the control unit 22 is configured to actuate the stimulation unit 12 to successively generate the stimulus Sⱼ and its intra-channel preceding stimulus S_{ic} via the same channel cⱼ in a sequence such that the minimum intra-channel non-stimulation period P_{ic} is provided between the stimulus Sⱼ and its intra-channel preceding stimulus S_{ic}. Further, the control unit 22 is configured to prevent the stimulation unit 12 from generating stimuli via the stimulation channel used for generating the stimulus Sⱼ and the intra-channel preceding stimulus S_{ic} during a period provided between the stimulus Sⱼ and the intra-channel preceding stimulus S_{ic}.

In step S7, the stimulus Sⱼ is included into the stimulation pattern. Then, the process proceeds to step S8 before returning to step S1, thereby starting a new iteration.

In the shown procedure, the control unit 22 may be configured to set the duration of the minimum cross-channel non-stimulation period P_{cc} in the range of 1/3 to 3/2 times a stimulus duration of the preceding stimulus S_{ic}, in particular when the stimulus Sⱼ and its preceding stimulus Sⱼ₋₁ are generated via different stimulation channels. Further, the control unit may be configured to set the duration of the minimum intra-channel non-stimulation period P_{ic} in the range of 3 to 5 times the stimulus duration of the intra-channel preceding stimulus S_{ic}.

In the described procedure, the control unit 22 is configured to provide one stimulus per actuation interval. In a further development, the control unit 22 may be configured to generate more than one stimulus during an actuation interval. Specifically, the control unit 22 may be configured to set a number nⱼ of stimuli to be simultaneously generated during an actuation interval Iⱼ, wherein the number nⱼ may be variedly set within the sequence to further increase randomness in the channel domain, e.g., by employing an exponential distribution process and/or a Markov process and/or any other suitable stochastic or deterministic or combined stochastic-deterministic process. A set of stimuli to be generated simultaneously during actuation interval Iⱼ may be denoted as \vec{S}ⱼ and may be expressed as:

$${\vec{S}}_{j} = \{ {S}_{j \_ 1} , … , {S}_{j\_n} \} \qquad ( 6 )$$

In this configuration, for each actuation interval Iⱼ, steps S4 to S7 may be iteratively performed nⱼ times, i.e. for each stimulus S_{j_n} included in the set of stimuli \vec{S}ⱼ, respectively.

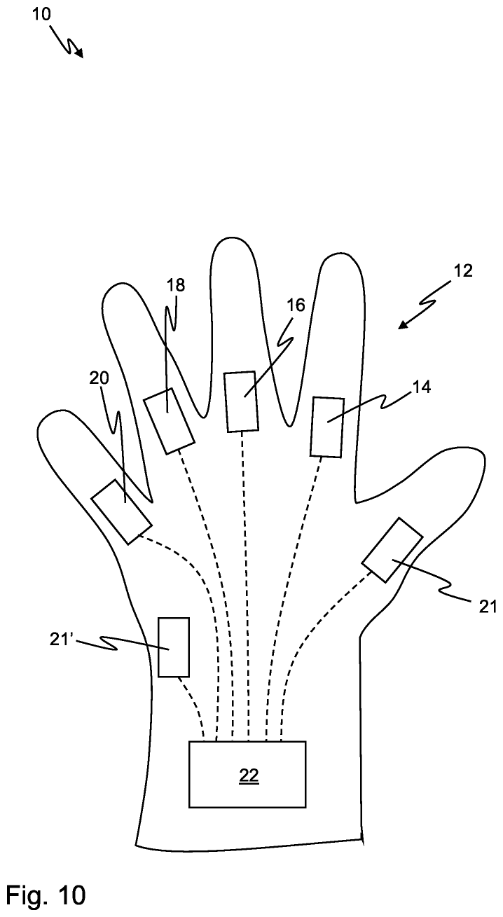

FIG. 10 depicts a further embodiment of the medical device 10 in the form of a therapeutic glove being equipped with a stimulation unit 12, the configuration and arrangement of which differs from the device depicted in FIG. 1. Specifically, as can be gathered from FIG. 10 depicting an upper side of the therapeutic glove, the device 10 comprises stimulation elements 14 to 20 configured for acting upon proximal phalanges of the patient's fingers, in particular at the back of the fingers. Additionally, the device comprises further stimulation elements 21, 21′ remotely placed from the stimulation elements 14-20. Specifically, one of the further stimulation elements, i.e. stimulation element 21, acts upon the thumb and the other, i.e. stimulation element 21′, acts upon the back of the hand. By such a configuration, the fingertips are free, thereby rendering the device more convenient.

The control unit 22, as set forth above, is configured to determine a minimum non-stimulation period between stimuli, wherein the duration of the minimum non-stimulation period is determined in dependence on the stimulus characteristics of the stimuli between which the minimum non-stimulation period is to be provided. Each stimulus when being administered to the patient's body is configured to stimulate a target neuronal population. It has been found that the closer the target neuronal populations stimulated by two successive stimuli are located, the longer the minimum non-stimulation period between these two stimuli is to be set in order to effectively avoid mutual masking and habituation effects. Thus, when determining the duration of the minimum non-stimulation period between a first and a second stimuli, the control unit 22 is configured to take into account the target neuronal population to be stimulated by the two stimuli. In other words, the control unit 22 is configured to set the duration of the minimum non-stimulation period between a first and a second stimulus in dependence on the target neuronal populations to be stimulated by the first and the second stimulus.

It has been further found that the stimulation channel via which the respective stimuli are generated are associated to and thus indicative of the target neuronal population to be stimulated thereby. Thus, the control unit is configured to set the duration of the minimum non-stimulation period between a first and a second stimulus in dependence on their stimulus channel, i.e. the stimulation element via which the respective stimuli are generated. Specifically, the control unit 22 is configured to set a longer duration of the minimum non-stimulation period between a first and a second stimulus if the stimulation elements via which these stimuli are generated are arranged further apart. In respect of the embodiment depicted in FIG. 10, the control unit 22 is configured to set the duration of the minimum non-stimulation period between stimuli generated by neighboring stimulation elements such that it is longer compared to a non-stimulation period between stimuli generated by stimulation elements located further apart. As a result, the control unit 22 may be configured to set the duration of the non-stimulation period between respective stimuli as depicted in Table 1.

Specifically, in Table 1, each column refers to a stimulation element generating a first stimulus and each row refers to a stimulation element generating a subsequent second stimulus. Accordingly, each cell of the table is indicative of the duration of the minimum non-stimulation period to be provided between the first and the second stimulus. Specifically, each cell defines the duration of the minimum non-stimulation period with respect to the duration of the first stimulus, i.e. by indicating whether the duration of the minimum non-stimulation period is 5 or 3 or 0.4 or 0 times the duration of the first stimulus.

By such a configuration, the control unit 22 defines the duration between successive stimuli by taking into account the respective target neuronal population to be stimulated by these stimuli. In this way, the control unit may avoid that stimuli, which may stimulate overlapping or neighboring neuronal populations, are generated too quickly one after the other, thereby preventing masking and habituation effects.

In a further development, the control unit 22 may be configured to take into account stimulation channel specific propagation delays when actuating the stimulation unit 22, in particular when setting or providing the stimulation pattern. For doing so, the control unit 22 may be configured to associate to each stimulation channel 14-21 a channel specific propagation delay Dⱼ, wherein j indicates the associated stimulation channel. Accordingly, the control unit 22 may set an actuation onset aⱼ, i.e. the point in time at which actuation of a stimulation unit or element begins, according to the following equation:

$${a}_{j} , = {t}_{j} - {D}_{j} , \qquad ( 7 )$$

wherein tⱼ denotes the stimulus onset, i.e. the point in time at which delivery of a stimulus is to begin and Dⱼ denotes the channel specific propagation delay for stimulation channel j.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

### LIST OF REFERENCE NUMERALS

10 medical device
12 stimulation unit
14-21 stimulation channel, stimulation element
22 control unit
α actuation onset
D channel specific propagation delay
I actuation interval
j sequence number
t stimulus onset
P_{cc} minimum cross-channel non-stimulation period
P_{ic} minimum intra-channel non-stimulation period
S stimuli
T time interval between successive time onsets

1. A medical device for stimulating neurons of a patient to suppress a pathologically synchronous activity of the neurons, the device comprising:
a non-invasive stimulation unit configured for administering different stimuli (s) to a body of a patient via at least one stimulation channel; and
a control unit for selectively actuating the stimulation unit, the control unit being configured to actuate the stimulation unit to successively generate a first stimulus (sⱼ₋₁; s_{ic}) and a subsequent second stimulus (sⱼ) in a sequence such that a minimum non-stimulation period (P_{cc}, P_{ic}) is provided between the first and the second stimulus (sⱼ₋₁, sⱼ; s_{ic}, sⱼ),
wherein the control unit is further configured to set a duration of the minimum non-stimulation period (P_{cc}, P_{ic}) in dependence on at least one stimulus characteristic of the first stimulus (sⱼ₋₁; s_{ic}).

2. The medical device according to claim 1, wherein the stimulation unit is configured to administer at least one of tactile stimuli, vibratory stimuli, vibrotactile stimuli, visual stimuli, electrical stimuli, thermal stimuli, acoustic stimuli, olfactory stimuli and magnetic stimuli.

3. The medical device according to claim 1, wherein the stimulation unit is configured to generate the different stimuli (s) via at least two stimulation channels.

4. The medical device according to claim 1, wherein the stimulation unit comprises at least two stimulation elements which are separately and selectively actuatable by the control unit to generate the different stimuli (s), wherein each stimulation channel is associated to at least one stimulation element.

5. The medical device according to claim 4, wherein the stimulation elements are configured to, in a state in which the stimulation elements are attached to or interact with the body of the patient, deliver stimuli (s) to different anatomic areas of the body of the patient.

6. The medical device according to claim 1, wherein the stimulus characteristic comprises at least one of a stimulus modality, a stimulus frequency, a stimulus amplitude, a stimulus waveform, a stimulus duration, and stimulus channel.

7. The medical device according to claim 1, wherein the stimulus characteristic specifies or is indicative of a psychophysical strength of the stimuli (s).

8. The medical device according to claim 1, wherein the control unit is configured to generate the first stimulus (sⱼ₋₁; s_{ic}) and the second stimulus (sⱼ) via one stimulation channel or via different stimulation channels.

9. The medical device according to claim 1, wherein the control unit is configured to prevent the stimulation unit from generating stimuli via the at least one stimulation channel used for generating the first stimulus (sⱼ₋₁; s_{ic}) and the second stimulus (sⱼ) during a period provided between the first stimulus (sⱼ₋₁; s_{ic}) and the second stimulus (sⱼ).

10. The medical device according to claim 1, wherein the control unit is configured to set the duration of the minimum non-stimulation period (P_{cc}, P_{ic}) in dependence on at least one of a stimulus channel of both the first and the second stimulus (sⱼ₋₁, sⱼ; s_{ic}, sⱼ), a stimulus frequency of both the first and the second stimulus (sⱼ₋₁, sⱼ; s_{ic}, sⱼ) and a stimulus duration of the first stimulus (sⱼ₋₁; s_{ic}).

11. The medical device according to claim 1, wherein the control unit is configured to set a duration of the minimum non-stimulation period to a first duration (P_{cc}) when the first and the second stimulus (sⱼ₋₁, sⱼ) are generated via different stimulation channels and to set the duration of the minimum non-stimulation period to a second duration (P_{ic}) being longer than the first duration (P_{cc}) when the first and the second stimulus (sⱼ₋₁, sⱼ) are generated via one stimulation channel.

12. The medical device according to claim 1, wherein the control unit is configured to set the duration of the minimum non-stimulation period in the range of 0.25 to 3 times a stimulus duration of the first stimulus (sⱼ₋₁) when the first and the second stimulus (sⱼ₋₁, sⱼ) are generated via different stimulation channels and to set the duration of the minimum non-stimulation period in the range of 3 to 5 times the stimulus duration of the first stimulus (s_{ic}) when the first and the second stimulus (s_{ic}, sⱼ) are generated via one stimulation channel.

13. The medical device according to claim 1, wherein in order to provide the minimum non-stimulation period between the first and the second stimulus (sⱼ₋₁, sⱼ; s_{ic}, sⱼ), the control unit is configured to:
shorten a stimulus duration of the first stimuli (sⱼ₋₁, s_{ic}), or
adapting a stimulus frequency of the first or the second stimulus (sⱼ₋₁, sⱼ; s_{ic}, sⱼ), such that the stimulus frequency of the first and the second stimuli (sⱼ₋₁, sⱼ; s_{ic}, sⱼ) differ, or
adapting the stimulus channel of the first or the second stimulus (sⱼ₋₁, sⱼ; s_{ic}, sⱼ) such that the stimulation channel via which the first and the second stimulus (sⱼ₋₁, sⱼ; s_{ic}, sⱼ) are to be delivered differ from one another.

14. The medical device according to claim 1, wherein the control unit is configured to generate the first and the second stimulus (s_{ic}, sⱼ) via a first stimulation channel and to generate a third stimulus (sⱼ₋₁) via a second stimulation channel which is to be generated prior to the second stimulus (sⱼ), such that a further minimum non-stimulation period (P_{cc}) is provided between the third and the second stimulus (sⱼ₋₁, sⱼ), wherein the control unit is configured to set the duration of the further minimum non-stimulation period (P_{cc}) in dependence on at least one stimulus characteristic of the third stimulus (sⱼ₋₁).

15. The medical device according to claim 14, wherein the control unit is configured to set a duration of the further minimum non-stimulation period (P_{cc}) in dependence on a stimulus channel of both the third and the second stimulus (sⱼ₋₁, sⱼ).
